(19) United States
(12) Patent Application Publication
(10) Pub. No.: US 2003/0152973 A1
(43) Pub. Date: Aug. 14, 2003

(54) G-protein coupled receptor org3

(76) Inventors: Morag A Grassie, Glasgow (GB); Derek Gatherer, Glasgow (GB); Colin Andrew McLean Semple, Edinburgh (GB)

Correspondence Address: William M Blackstone, Akzo Nobel, Patent Department Intervet Inc, 405 State Street, Millsboro, DE, 19966, US

(21) Appl. No.: 10/296,121

(22) PCT Filed: May 21, 2001
(86) PCT No.: PCT/EP01/05919

(30) Foreign Application Priority Data
May 24, 2000 (EP) 00304419.5

(51) Int. Cl. C12Q 1/68; C07H 21/04; C07K 14/47; C12P 21/02; C12N 5/06

(52) U.S. Cl. 435/6; 435/69.1; 435/320.1; 435/325; 530/350; 536/23.2

(57) ABSTRACT

The present invention provides a full length cDNA sequence that codes for a G-protein coupled receptor, as well as the complete gene and the encoded protein. The present invention provides a recombinant cell line expressing these receptors at appropriate levels such that novel compounds active at these receptors may be identified for therapeutic use. The receptor sequence described in this invention is a member of a novel GPCR receptor sub-family which has no known endogenous ligand. This cDNA can be used to identify novel compounds active at the receptor for therapeutic intervention especially in the field of CNS disorders, more in particular for the treatment of bipolar affective disorder (BPAD). The nucleotide sequence of this gene could he used for diagnostic purposes in psychiatric patients and susceptible populations.

The present invention provides a full length cDNA sequence that codes for a G-protein coupled receptor, as well as the complete gene and the encoded protein. The present invention provides a recombinant cell line expressing these receptors at appropriate levels such that novel compounds active at these receptors may be identified for therapeutic use. The receptor sequence described in this invention is a member of a novel GPCR receptor sub-family that has no known endogenous ligand. This cDNA can be used to identify novel compounds active at the receptor for therapeutic intervention especially in the field of CNS disorders, more in particular for the treatment of bipolar affective disorder (BPAD). The nucleotide sequence of this gene could be used for diagnostic purposes in psychiatric patients and susceptible populations.

The G-protein coupled receptor (GPCR) superfamily is one of the largest protein families identified to date. This family comprises over 800 cloned members from a wide range of species, and includes at least 300 human members. GPCRs have a proven history as excellent therapeutic targets with between 40-50% of drug targets to date being GPCRs (Murphy, 1998). GPCRs are responsive to a wide variety of stimuli and chemical transmitters, including light, biogenic amines, amino acids, peptides, lipids, nucleosides, and large polypeptides. This results in the regulation of multiple processes including neurotransmission, cellular metabolism, secretion, cellular differentiation and growth as well as inflammatory and immune responses. Many of these GPCRs are expressed in the brain and may be exploited as therapeutic targets for the treatment of CNS disorders. More significantly, many GPCRs with no known endogenous ligands are still being identified in the public and proprietary databases. These orphan GPCRs represent potential novel therapeutic targets for a range of therapeutic intervention and the treatment of a variety of disorders

Reverse pharmacology or functional genomics is currently being adopted within the drug discovery process. This is gene-based biology which aims to pharmacologically validate novel genes by either identifying surrogate ligands or their endogenous ligand.

There is evidence to suggest that in addition to novel orphan GPCRs, there also exist novel GPCR gene sub-families that bind previously unidentified ligands. Because many orphan GPCRs await to be assigned a natural ligand, many of these receptors may bind novel ligands which have not thus far been identified. (Civelli et al, 1999).

Orphan GPCRs are predicted to bind ligands, as it is postulated that inactive receptors should have been evolutionary discarded. Orphan receptors may therefore be used as baits to isolate their natural ligands or surrogate ligands. The use of this strategy in identifying novel ligands is exemplified in the identification of orphanin/nociceptin, orexins/hypocretins and prolactin-releasing peptide (Reinscheid et al 1995, Sakurai et al 1998, and Hinuma et al, 1998).

Many known G protein coupled receptors (GPCRs) are well established drug targets with a significant number of currently available drugs targeting such GPCRs (Wilson et al, 1998). Following activation of a GPCR by ligand binding to the receptor, the signal is amplified through a range of signal transduction cascades and consequently, regulation of this signal transduction pathway via a ligand binding to a GPCR offers the facility to modulate a tightly controlled biological pathway.

GPCRs mediate a wide range of biologically relevant processes and are responsive to a wide variety of stimuli and chemical/neurotransmitters, including light, biogenic amines, amino acids, peptides, lipids, nucleosides, and large polypeptides. How the cloning of a particular receptor has led to the development of a therapeutic compound is particularly exemplified in the case of the serotonin/adrenergic receptor. Additionally a number of diseases are reported to be associated with mutations in known GPCRs (Wilson et al, 1998). The signalling pathways that mediate the actions of GPCRs have also been implicated in many biological processes significant to the pharmaceutical industry. Such signalling pathways involve G proteins, second messengers such as cAMP or calcium (Lefkowitz, 1991), effector proteins such as phospholipase C, adenylyl cyclase, RGS proteins, protein kinase A and protein kinase C (Simon et al, 1991).

For example a GPCR can be activated by a ligand, binding to the receptor resulting in the activation of a G protein which conveys the message onto the next component of the signal transduction pathway. Such a component could be adenylyl cyclase. In order for activation of this enzyme, the relevant G protein, of which there is a family, must exchange GTP for GDP, which is bound when the G protein is in an inactive state. The exchange of GDP for GTP can occurs following the binding of ligand to the GPCR, however, some basal exchange of GDP for GTP can also occur depending on the receptor under investigation.

The conversion of GTP bound at the G protein to GDP occurs by hydrolysis and is catalysed by the G protein itself. Following this hydrolysis the G protein is returned to its inactive state. Consequently, the G protein mediates the transfer of the signal from the activated receptor to the intracellular signalling pathway, but also introduces an additional level of control, by controlling the length of time which the receptor can activate the intracellular signalling pathway through the GTP bound G protein.

In general the topology of these receptors is such that they contain 7 transmembrane domains consisting of approximately 20-30 amino acids. Consequently, these receptors are frequently known as 7TM receptors. These 7TM domains can be defined by consensus amino acid sequences and by structural prediction algorithms such as the Kyte Doolittle programme. Within the putative transmembrane domains, hydrophobic helixes are formed which are connected via extracellular and intracellular loops. The N-terminal end of the polypeptide is on the exterior face of the membrane with the C-terminal on the interior face of the membrane.

A number of additional features are frequently observed in GPCRs. These include glycosylation of the N-terminal tail. A conserved cysteine in each of the first two extracellular loops, which are modified such that disulphide bonds are formed, which is believed to result in a stabilised functional tertiary structure. Other modifications which occur on GPCRs include lipidation (eg palmitoylation and farnesylation) and phosphorylation often in the C terminal tail. Most GPCRs also have sites for phosphorylation in the third intracellular loop, a region, which is believed to contribute to G protein interactions and signal transduction. Phosphorylation of the third intracellular loop by specific receptor kinases such as cAMP dependent protein kinase(cAPK) or a class of GPCR kinases (GRKs) in several GPCRs such as β-adrenoreceptor also mediates in the desensitization of such a receptor. Consequently, specific mutations in particular regions of the GPCR can have functional significance. GRKs are known to phosphorylate GPCRs on multiple sites with theronine and serine residues as targets. The phosphorylation not only inactivates the receptor but also allows the receptor with an additional inhibitory protein known as β-arrestin. This interaction can also be used as an indication that the GPCR in question has been activated.

Although as yet only limited three-dimensional crystal structure data is available for GPCRs some details of the ligand binding site present on GPCRs has been reported. For some receptors the ligand binding sites are believed to comprise hydrophilic pockets formed by some of the transmembrane domains. Within the transmembrane domain, the amino acid within the α-helical structure align themselves such that the hydrophilic surface of the amino acid is facing inwards towards the centre of the ligand binding pocket. This results in a postulate polar ligand binding site. The third transmembrane domain of has been reported to be involved in ligand binding in several GPCRs. In particular the aspartate of TM3, serines of TM5, asparagine of TM6 and phenylalanine or tyrosines of TM6 and/or TM7 have been implicated in ligand binding.

In addition to activating intracellular signalling pathways, GPCRs can also couple via G proteins to additional gene families such as ion channels, transporter and enzymes. Many GPCRs are present in mammalian systems exhibiting a range of distribution patterns from very specific to very widespread. For this reason following the identification of a putative novel GPCR by bioinformatics, assigning a therapeutic application to the novel GPCR is not obvious due to this diverse function and distribution of previously reported GPCRs.

There is clearly a need to identify and characterise novel GPCRs that can function to alter disease status either correction, prevention or amelioration. Such disease are diverse and include but are not exclusive to depression, schizophrenia, anxiety, neurological disorders, obesity, insomnia, addiction, neurodegeneration, hypotension, hypertension, acute heart failure, athrothrombosis, athrosclerosis, osteoporosis and rheumatoid arthritis.

BPAD is a psychiatric illness showing a combination of depression and elevated mood in cycles (manic-depression). BPAD is familial so has a degree of genetic etiology, with the estimated lifetime risk of developing BPAD is 0.8%. Blackwood et al, (1996) showed linkage on 4p16 in a bipolar family by genome wide scan (193 markers). Marker D4S394 gave Logarithm of Odds Ratio (LOD) score of 4.1. A LOD score greater than 4 indicates that there is only a 1 in 10 000 probability that the finding happened by chance, and therefore a LOD score of 4.1 is highly significant. Three point analyses gave LOD of 4.8 between markers D4S431 and D4S403. A further eleven families showed linkage to D4S394 with LOD 4.1. These data were strengthened by Visscher et al, (1999) who detecting Quantitative Trait Loci (QTL; ie those genetic factors that generate continuously variable, measurable phenotypes) for uni- and bipolar disorder in the same region and the same initial family. Here a QTL (accounting for 25% of the trait—uni- or bipolar affective disorder) was observed with a linkage LOD of 5.9 over the 10centimorgan (cM) region 4p region identified by Blackwood et al. Several other groups have replicated the findings of a BPAD linked region on 4p (Ewald et al, 1998, Detera-Wadleigh et al 1997; Asherson et al 1998; Kennedy & Macciardi 1998). Ginns et al (1996), found a protective effect or “wellness” gene at the same locus on 4p in an Amish population. Several other loci have given positive LOD scores for BPAD, however none have the same significance or replication as the 4p16 findings. Therefore, the genetic linkage evidence is strong for a BPAD disease locus on 4p16.

The present invention provides a brain expressed gene/protein which we termed ORG3 and which was found to be located in the above described 4p16 linked region. Analysis of the gene provides evidence that it is a GPCR. The gene may therefore be used in conventional expression systems in order to select compounds that specifically react with ORG3. These compounds may then be used to treat BPAD.

The present invention relates to ORG3, in particular ORG3 polypeptides, ORG3 polynucleotides, recombinant materials and methods of their production. Additionally the invention relates to methods which for such polypeptides and polynucleotides can be used to identify compounds such as agonists or antagonists active at the invention for treatment of disease, such as psychiatric diseases but in particular bipolar and unipolar disorders, schizophrenia and anxiety. Use of agonists or antagonists active at the said invention may be used to correct diseases associated with an imbalance of ORG3 and associated pathways. In particular, this invention relates to a diagnostic assay for identifying modifications in ORG3 gene or expression associated with CNS diseases and especially preferred for bipolar depression and affective disorders.

The complete cDNA sequence of ORG3 shown in SEQ ID NO: 2 was translated which resulted in the amino acid sequence of SEQ ID NO: 3 which was then compared with known protein sequences. The closest match was g6644328 rat orphan G protein-coupled receptor GPR26. This shows only 50% homology at the amino acid level indicating that these are truly different receptors however, they may belong to the same receptor sub-family.

ORG3 also has a high degree of homology with human sequence flh2882 from patent application number WO 9937679. There was no close match to any other human sequence. The genomic sequence of ORG3 is provided in SEQ ID NO: 1. ORG3 is a member a novel receptor sub-family of GPCR receptors, that includes the orphan receptors GPR26 (Lee et al, 2000), and more distantly SREB1 (patent WO9946378_A1) and SREB 2 (patent WO9946378-A1).

ORG3 is predominantly expressed in the brain and could hardly if at all be detected in any other tissue.

The genomic polynucleotides of the present invention may be obtained using standard cloning and screening techniques from a human genomic DNA library, however a full length cDNA product lacking the non-coding region of the invention as described in SEQ ID NO:1 can only be obtained from a cDNA library such as a brain cDNA library. Polynucleotides detailed in this invention could also be generated from genomic DNA or synthesized using well known and commercially available techniques.

As GPCRs have key sequence motifs, aligning the 7 transmembrane domains of orphan and non-orphan GPCRs can create a phylogenetic tree. This tree groups many of the well characterised GPCRs into families such as biogenic amine, chemokine, purinergic, olfactory, angiotensin, neuropeptide, opioid, etc. The placement of novel orphan GPCRs on this tree allows a prediction to be made as to the sub-family to which this GPCR belongs and possible biochemical function. ORG3 is in a family with GPR26, SREB1 and SREB2. These GPCRs are expressed predominantly in brain and thus ORG3 may have relevance to CNS disorders.

In order to determine which GPCRs ORG3 was most closely related to, phylogenetic analysis of its amino acid sequence was performed. Transmembrane regions were identified by hydrophobicity plot and/or alignment with known transmembrane regions. Concatenated transmembrane regions were aligned (HMMAlign) and trees were generated by ProtPars and viewed in TreeView. This determination led to the conclusion that ORG3 lies in the receptor family that also includes GPR26, and more distantly SREB1 and SREB2.

The sequences of the present invention can be used to derive primers and probes for use in DNA amplification reactions in order to perform diagnostic procedures or to identify further, neighbouring genes which also may contribute to the development of CNS disorders.

It is known in the art that genes may vary within and among species with respect to their nucleotide sequence. The ORG3 genes from other species may be readily identified using the above probes and primers. Therefore, the invention also comprises functional equivalents, which are characterised in that they are capable of hybridising to at least part of the ORG3 sequence shown in SEQ ID NO: 1, preferably under high stringency conditions.

Two nucleic acid fragments are considered to have hybridisable sequences if they are capable to hybridising to one another under typical hybridisation and wash conditions, as described, for example in Maniatis, et al., pages 320-328, and 382-389, or using reduced stringency wash conditions that allow at most about 25-30% basepair mismatches, for example: 2×SSC, 0.1% SDS, room temperature twice, 30 minutes each, then 2×SSC, 0.1% SDS 37° C. once, 30 minutes; then 2×SSC, room temperature twice ten minutes each. Preferably, homologous nucleic acid strands contain 15-25% basepair mismatches, even more preferably 5-15% basepair mismatches. These degrees of homology can be selected by using wash conditions of appropriate stringency for identification of clones from gene libraries or other sources of genetic material, as is well known in the art.

Furthermore, to accommodate codon variability, the invention also includes sequences coding for the same amino acid sequences as the sequences disclosed herein. Also portions of the coding sequences coding for individual domains of the expressed protein are part of the invention as well as allelic and species variations thereof. Sometimes, a gene expresses different isoforms in a certain tissue which includes splicing variants, that may result in an altered 5′ or 3′ mRNA or in the inclusion of an additional exon sequence. Alternatively, the messenger might have an exon less as compared to its counterpart. These sequences as well as the proteins encoded by these sequences all are expected to perform the same or similar functions and form also part of the invention.

The sequence information as provided herein should not be so narrowly construed as to require inclusion of erroneously identified bases. The specific sequence disclosed herein can be readily used to isolate further genes which in turn can easily be subjected to further sequence analyses thereby identifying sequencing errors. Thus, in one aspect, the present invention provides for isolated polynucleotides encoding a novel gene, disrupted in psychiatric disease in particular bipolar affective disorder.

The DNA according to the invention may be obtained from cDNA. Alternatively, the coding sequence might be genomic DNA, or prepared using DNA synthesis techniques. The polynucleotide may also be in the form of RNA. The polynucleotide may be in single stranded or double stranded form. The single strand might be the coding strand or the non-coding (anti-sense) strand.

The present invention further relates to polynucleotides which have at least 80%, preferably 90% and more preferably 95% and even more preferably at least 98% identity with SEQ ID NO:1. Such polynucleotides encode polypeptides which retain the same biological function or activity as the natural, mature protein.

The percentage of identity between two sequences can be determined with programs such as DNAMAN (Lynnon Biosoft, version 3.2). Using this program two sequences can be aligned using the optimal alignment algorithm of Smith and Waterman (1981). After alignment of the two sequences the percentage identity can be calculated by dividing the number of identical nucleotides between the two sequences by the length of the aligned sequences minus the length of all gaps.

The DNA according to the invention will be very useful for in vivo or in vitro expression of the novel gene according to the invention in sufficient quantities and in substantially pure form.

In another aspect of the invention, there are provided polypeptides comprising the amino acid sequence encoded by the above described DNA molecules.

Preferably, the polypeptides according to the invention comprise at least part of the amino acid sequences as shown in SEQ ID NO: 3.

Also functional equivalents, that is polypeptides homologous to SEQ ID NO: 3 or parts thereof having variations of the sequence while still maintaining functional characteristics, are included in the invention.

The variations that can occur in a sequence may be demonstrated by (an) amino acid difference(s) in the overall sequence or by deletions, substitutions, insertions, inversions or additions of (an) amino acid(s) in said sequence. Amino acid substitutions that are expected not to essentially alter biological and immunological activities, have been described. Amino acid replacements between related amino acids or replacements which have occurred frequently in evolution are, inter alia Ser/Ala, Ser/Gly, Asp/Gly, Asp/Asn, lle/Val (see Dayhof, M.D., Atlas of protein sequence and structure, Nat. Biomed. Res. Found., Washington D.C., 1978, vol. 5, suppl. 3). Based on this information Lipman and Pearson developed a method for rapid and sensitive protein comparison (Science, 1985, 227, 1435-1441) and determining the functional similarity between homologous polypeptides. It will be clear that also polynucleotides coding for such variants are part of the invention.

The polypeptides according to the present invention include the polypeptides comprising SEQ ID NO:3 but also their isoforms, i.e. polypeptides with a similarity of 70%, preferably 90%, more preferably 95%. Also portions of such polypeptides still capable of conferring biological effects are included. Especially portions which still bind to ligands form part of the invention. Such portions may be functional per se, e.g. in solubilized form or they might be linked to other polypeptides, either by known biotechnological ways or by chemical synthesis, to obtain chimeric proteins. Such proteins might be useful as therapeutic agent in that they may substitute the gene product in individuals with aberrant expression of the ORG3 gene.

The sequence of the gene may also be used in the preparation of vector molecules for the expression of the encoded protein in suitable host cells. A wide variety of host cell and cloning vehicle combinations may be usefully employed in cloning the nucleic acid sequence coding for the ORG3 gene of the invention or parts thereof. For example, useful cloning vehicles may include chromosomal, non-chromosomal and synthetic DNA sequences such as various known bacterial plasmids and wider host range plasmids and vectors derived from combinations of plasmids and phage or virus DNA.

Vehicles for use in expression of the genes or a ligand-binding domain thereof of the present invention will further comprise control sequences operably linked to the nucleic acid sequence coding for a ligand-binding domain. Such control sequences generally comprise a promoter sequence and sequences which regulate and/or enhance expression levels. Of course control and other sequences can vary depending on the host cell selected.

Suitable expression vectors are for example bacterial or yeast plasmids, wide host range plasmids and vectors derived from combinations of plasmid and phage or virus DNA. Vectors derived from chromosomal DNA are also included. Furthermore an origin of replication and/or a dominant selection marker can be present in the vector according to the invention. The vectors according to the invention are suitable for transforming a host cell.

Recombinant expression vectors comprising the DNA of the invention as well as cells transformed with said DNA or said expression vector also form part of the present invention.

Suitable host cells according to the invention are bacterial host cells, yeast and other fungi, plant or animal host such as Chinese Hamster Ovary cells or monkey cells. Thus, a host cell which comprises the DNA or expression vector according to the invention is also within the scope of the invention. The engineered host cells can be cultured in conventional nutrient media which can be modified e.g. for appropriate selection, amplification or induction of transcription. The culture conditions such as temperature, pH, nutrients etc. are well known to those ordinary skilled in the art.

The techniques for the preparation of the DNA or the vector according to the invention as well as the transformation or transfection of a host cell with said DNA or vector are standard and well known in the art, see for instance Sambrook et al., Molecular Cloning: A laboratory Manual. 2^{nd} Ed., Cold Spring Harbor Laboratory, Cold Spring Harbor, N.Y., 1989.

The proteins according to the invention can be recovered and purified from recombinant cell cultures by common biochemical purification methods including ammonium sulfate precipitation, extraction, chromatography such as hydrophobic interaction chromatography, cation or anion exchange chromatography or affinity chromatography and high performance liquid chromatography. If necessary, also protein refolding steps can be included.

ORG3 gene products according to the present invention can be used for the in vivo or in vitro identification of novel ligands or analogs thereof. For this purpose binding studies can be performed with cells transformed with DNA according to the invention or an expression vector comprising DNA according to the invention, said cells expressing the ORG3 gene products according to the invention.

Alternatively also the ORG3 gene products according to the invention as well as ligand-binding domains thereof can be used in an assay for the identification of functional ligands or analogues for the ORG3 gene products.

Methods to determine binding to expressed gene products as well as in vitro and in vivo assays to determine biological activity of gene products are well known. In general, expressed gene product is contacted with the compound to be tested and binding, stimulation or inhibition of a functional response is measured.

Thus, the present invention provides for a method for identifying ligands for ORG3 gene products, said method comprising the steps of:

a) introducing into a suitable host cell a polynucleotide according to the invention,

b) culturing cells under conditions to allow expression of the DNA sequence

c) optionally isolating the expression product

d) bringing the expression product (or the host cell from step b) into contact with potential ligands which will possibly bind to the protein encoded by said DNA from step a);

e) establishing whether a ligand has bound to the expressed protein.

f) Optionally isolating and identifying the ligand

As a preferred way of detecting the binding of the ligand to the expressed protein, also signal transduction capacity may be measured.

The present invention thus provides for a quick and economic method to screen for therapeutic agents for the prevention and/or treatment of diseases related to CNS disorders. The method is especially suited to be used for the high throughput screening of numerous potential ligands.

Compounds which activate or inhibit the function of ORG3 gene products may be employed in therapeutic treatments to activate or inhibit the polypeptides of the present invention.

Also within the scope of the invention are antibodies, especially monoclonal antibodies raised against the polypeptide molecule according to the invention. Such antibodies can be used therapeutically to inhibit ORG3 gene product function and diagnostically to detect ORG3 gene products.

The invention furthermore relates to the use of the ORG3 gene products as part of a diagnostic assay for detecting psychiatric abnormalities or susceptibility to psychiatric disorders related to mutations in the nucleic acid sequences encoding the ORG3 gene. Such mutations may e.g. be detected by using PCR (Saiki et al., 1986,). Also the relative levels of RNA can be determined using e.g. hybridization or quantitative PCR technology. The presence and the levels of the ORG3 gene products themselves can be assayed by immunological technologies such as radioimmuno assays, Western blots and ELISA using specific antibodies raised against the gene products. Such techniques for measuring RNA and protein levels are well known to the skilled artisan.

The determination of expression levels of the ORG3 gene products in individual patients may lead to fine tuning of treatment protocols.

Also, transgenic animals may be prepared in which the expression of the ORG3 gene is altered or abolished and includes the use of such an animal, as an in vivo animal model for psychiatric diseases.

### EXAMPLE 1

Full-Length Sequence Identification

Hidden Markov Modelling on the Human Genome Project high-throughput genomic clones in EMBL database release 60, predicted a GPCR protein fragment on BAC clone AC007104. This sequence was extended in N-terminal and C-terminal direction until a plausibly full-length receptor protein sequence was determined. The predicted cDNA and the intron-exon boundaries were identified by comparing the predicted protein sequence with the genomic DNA. At the time of identification, no expression information from the proprietary database or public databases, was available for the novel protein.

The presence of the complete ORG3 cDNA in human brain was confirmed by PCR using primers designed against the predicted sequence encompassing the ATG translation initiation site and the TGA stop codon (SEQ ID NO: 2). Each PCR reaction contained 1× PCR buffer (Expand High Fidelity buffer), 1.5 mM MgCl₂, 5 μl human whole brain Marathon-Ready cDNA (Clontech), 1 μM primer 1 and 2 (ORG3 forward primer 5′-CCA CCA TGG GCC CCG GCG AGG CGC TGC T 3′ and ORG3 reverse primer 5′-TCA GTG TGT CTG CTG CAG GCA GGA ATC 3′,), 400 μM dATP, 400 μM dCTP, 400 μM dGTP, 400 μM dTTP, 10% DMSO and 2.625 units Expand High Fidelity PCR enzyme mix in a total volume of 50 μl. Reactions were cycled in a MJ Research PTC-200 Thermal Cycler using the following conditions: 95° C., 5 min and 40 cycles of 95° C. for 1 min, 58° C. for 1 min 30 sec, 72° C. for 2 min, followed by an extension of 72° C. for 10 min. PCR products of approximately 1.1 Kb were identified, purified and sequenced using an ABl Prism 310 Genetic analyser (PE Biosystems). Sequencing reactions were performed using ABl Prism BigDye Terminator cycle sequencing Ready reaction kit (PE Biosystems). Each sequencing reaction contained 300 ng cDNA clone, 3.2 pmol sequencing primer, and PE Biosystems Terminator Ready reaction mix in a final volume of 20 μl. Reactions were cycled as follows: 25 cycles of 96° C. for 10 sec, 50° C. for 5 sec and 60° C. for 4 min in a PE Biosystems GeneAmp PCR system 9700. Following cycling, the extension products were precipitated by adding 2 μl 3M NaOAc (pH 4.6) and 50 μl 95% ethanol. Products were precipitated at RT for 15 min and collected by centrifugation at 14000 rpm for 20 min. Pellets were washed 2× with 70% ethanol prior to resuspension in 20 ul Template suppression reagent (PE Biosystems) for sequencing. The sequence clones encoded the entire human ORG3 open reading frame. The sequence is shown in SEQ ID NO: 2.

The full length sequence of ORG3 indicates that the cDNA consists of 1092 bp open reading frame (SEQ ID NO: 2) encoding an 363 amino acid protein (SEQ ID NO: 3).

### EXAMPLE 2

Tissue Distribution Analysis of ORG3 by PCR.

In order to further analyze the expression of the G-protein coupled receptor comprising SEQ ID NO: 1 in material from a variety of human tissues, PCR was performed using primers designed against the predicted ORG3 cDNA sequence (ORG3 forward primer 5′-CCA CCA TGG GCC CCG GCG AGG CGC TGC T 3′ (1-23) and ORG3 reverse primer 5′-TCA GTG TGT CTG CTG CAG GCA GGA ATC (1092-1065) 3′. Each PCR contained 1× PCR buffer, 1.5 mM Magnesium chloride, 200 μM dNTP mix, 1 μM each primer, 10% DMSO, 2.5 units Expand polymerase (Roche) and 5 μl human marathon ready cDNA (Clontech) in a total volume of 50 μl. The human cDNAs investigated for expression of ORG3 were: heart, kidney, skeletal muscle, spleen, ovary, lung, liver, thymus, testis, small intestine and brain (Clontech). A positive control reaction with human genomic DNA (Promega) was also set up. PCR amplification of the housekeeping gene G3PDH was performed as described above using sequence-specific primers purchased from Clontech, and this was used as a positive control for each cDNA template. Reactions were cycled in a MJ Research PTC-200 Thermal Cycler using the following conditions: 95° C., 5 min and 40 cycles of 95° C. for 1 min, 58° C. for 1 min 30 sec, 72° C. for 2 min, followed by an extension of 72° C. for 10 min. PCR products were separated on 1% agarose gels containing ethidium bromide (10 mg/ml) and visualised under UV light.

Following 40 cycles of amplification a faint band of 1092 bp corresponding to the full length ORG3 cDNA was observed only in brain, with a very faint band present in liver. Expression was virtually undetectable in the remainder of the peripheral tissues. The band detected in brain is consistent with the low level expression pattern observed for this orphan GPCR.

References

1. Asherson P, Mant R, Williams N, Cardno A, Jones L, Murphy K, Collier D A, Nanko S, Craddock N, Morris S, Muir W, Blackwood B, McGuffin P, Owen M J (1998) Mol Psychiatry 3(4):310-20.

2. Blackwood, D H, Morris,H L, et al, (1996) Nature Genetics 12, 427-30.

3. Civelli O, Reinscheid R K, Nothacker, H P. (1999) Brain Research 848:(2):63-65.

4. Detera-Wadleigh S D, Badner J A, Yoshikawa T et al (1997) Am. J. Med. Genet 74 (3) 254-262.

5. Ewald H, Degn B, Mors O, Kruse T A (1998). Mol Psychiatry Sep;3(5):442-8.

6. Ginns E I, St Jean P, Philibert R A (1998) PNAS 95(26):15531-6.

7. Hinuma, S., Habata, Y., Fujii, R., Kawamata, Y., Hosoya, M., Fukusumi, S., Kitada, C., Masuo, Y., Asano, T., Matsumoto, H., Sekiguchi, A., Kurokawa, T., Nishimura, O., Onda, M., Fujino, M. (1998). Nature 393:272-276.

8. Kennedy J L, Macciardi F M (1998) Psychiatr Genet 8(2):67-71.

9. Kyte J., Doolittle R. F. (1982) Biol. 157:105-132.

10. Lee,D. K., Lynch,K. R., Nguyen,T., Im,D.-S., Cheng,R., Saldivia,V. R., Liu,Y., Liu,I. S. C., Heng,H. H. Q., Seeman,P., George,S. R., O'Dowd,B. F. and Marchese,A. (2000) Biochim. Biophys. Acta 1490 (3), 311-323.

11. Lefkowitz, (1991) Nature, 351:353-354.

12. Murphy, A. J., Paul, J. I. & Webb, D. R. (1998) Drug Discovery & Development 1:192-199.

13. Reinscheid, R K., Nothaker, H. P., Bourson, A., Ardati, A., Henningensen, R. A., Bunzow, J. R., Grandy, D. K., Langen, H., Monsama, F. J., Civelli, O.(1995). Science 270:792-794.

14. Saiki et al., (1986) Nature 324: 163-166

15. Sakurai, T., Amemiya, A, Ishii, M., Matsuzaki, I., Chemeli, R. M., Tanaka, H., Williams, S. C., Richardson, J. A., Kozlowski, G. P., Wison, S., Arch, J. R. S., Buckingham, R. E., Haynes, A. G., Carr, S. A., Annan, R. S., McNulty, D. E., Liu, W. S., Terret, J. A., Elshourbagy, N. A., Bergsma, D. J., Yanagisawa, M (1998). Cell 92:573-585.

16. Simon et al.,(1991) Science, 252:802-808.

17. Smith & Waterman (1981) J. Mol. Biol. 147:195-197.

18. Visscher P M, Haley C S, Heath S C, Muir W J, Blackwood D H (1999) Psychiatric Genetics 9:75-84.

19. Wilson S, Bergsma D J, Chambers J K, Muir A I, Fantom K G, Ellis C, Murdock P R, Herrity N C, Stadel J M. (1998) Br J Pharmacol 125(7):1387-92.

1. A substantially pure polynucleotide, encoding the amino acid sequence of SEQ ID NO: 3 or its isoforms.

2. Polynucleotide according to claim 1, comprising the sequence according to SEQ ID NO: 2.

3. A recombinant expression vector comprising the polynucleotide according to claim 1 or 2 or fragments thereof.

4. A polypeptide according to SEQ ID NO: 3 or its isoforms.

5. Cell line transformed with a polynucleotide encoding at least part of the polypeptide according to claim 4

6. Cell line transformed with a polynucleotide according to claim 1 or 2 or fragments thereof or transformed with the expression vector of claim 3.

7. Cell line according to claim 6 of mammalian origin

8. Cell line according to claim 6 or 7 expressing an ORG3 gene product, wherein ORG3 gene is defined as a stretch of DNA hybridisable to the polynucleotide sequence according to SEQ ID NO: 1 and/or SEQ ID NO: 2

9. Use of a polynucleotide hybridisable to the ORG3 gene in the in vitro diagnosis of a psychiatric disorder, wherein ORG3 gene is defined as a stretch of DNA hybridisable to the polynucleotide sequence according to SEQ ID NO: 1 and/or SEQ ID NO: 2

10. Use of a cell line according to claim 6 to 8 in the in vitro diagnosis of a psychiatric disorder.

11. Use of a polypeptide encoded by a polynucleotide comprising SEQ ID NO 2 or fragments thereof in the in vitro diagnosis of a psychiatric disorder.

12. Use of a polynucleotide according to claims 1 or 2 or fragments thereof or the expression vector of claim 3 in a screening assay for the identification of new drugs.

13. Use of a polypeptide according to claim 4 or analogues or fragments thereof in a screening assay for the identification of drugs for the treatment of psychiatric disorders.

14. Use of a cell line according to claims 6 to 8 in a screening assay for the identification of new drugs for the treatment of psychiatric disorders.

15. A polynucleotide comprising SEQ ID NO 1 or fragments thereof for use as a medicament.

16. A polypeptide encoded by a polynucleotide comprising SEQ ID NO 2 or fragments thereof for use as a medicament

17. A polynucleotide comprising SEQ ID NO 2 or fragments thereof for use as a medicament for the treatment of a psychiatric disorder.

18. A polypeptide encoded by a polynucleotide comprising SEQ ID NO 2 or fragments thereof for use as a medicament for the treatment of a psychiatric disorder

19. Use of a polynucleotide comprising SEQ ID NO 2 or fragments thereof in the preparation of a medicament for the treatment of a psychiatric disorder

20. Use of a polypeptide encoded by a polynucleotide comprising SEQ ID NO 2 or fragments thereof in the preparation of a medicament for the treatment of a psychiatric disorder

21. Antibodies against the polypeptide according to claim 4

22. Method for the detection of a mutation in the ORG3 gene in a given subject comprising the steps of
a) providing a set of oligonucleotide primers capable of hybridising to the nucleotide sequence of the ORG3 gene
b) obtaining a sample containing nucleic acid from the subject
c) amplifying a region flanked by the primer set of step 1 using a nucleic acid amplification method
d) detecting whether the amplified region contains a mutation by
e) comparing the amplified sequence with the sequence of normal control subjects.
wherein ORG3 gene is defined as a stretch of DNA hybridisable to the polynucleotide sequence according to SEQ ID NO: 1.

23. A method for identifying ligands for ORG3 gene products, said method comprising the steps of:
a) introducing into a suitable host cell a polynucleotide according to claims 1 or 2 or an expression vector according to claim 3 or fragments thereof,
b) culturing cells under conditions to allow expression of the DNA sequence
c) optionally isolating the expression product
d) bringing the expression product (or the host cell from step b)) into contact with potential ligands which will possibly bind to the protein encoded by said DNA from step a);
e) establishing whether a ligand has bound to the expressed protein.
f) Optionally isolating and identifying the ligand

24. Compounds selected with a method according to claim 23 useful in the treatment of CNS disorders, in particular BPAD.

25. Use of compounds according to claim 24 for the manufacture of a medicament useful in the treatment of CNS disorders, in particular BPAD.


SEQUENCE LISTING (3 sequences)

SEQ ID NO: 1 - DNA, 7020 bp, Homo sapiens
ttatagcacgcccacgagtaccatatgccacacagggcacttccctacccactgtctcgaagcatcgccatgaccccagaacaagggtgaggaagctaaggttcagagaggaaaagccactgtcccggggccacccagcagccaggcagcccagccccgagctctcctccgccactactgctgtccacacccacatccggccctctcctcctcctcctcctgtacttcagccttagctcaaagtcaagccactggtgtctgggggtccccagtggcatctctgtggcagggccagtgccctttccacagggcttcttagaaggtgggggcgatgagccctgccaggccctcagtgtgtctgctgcaggcaggaatcattctctgtgtccacagagccgttgtgggtggacgctgggcgcggggttctcttcagcagctggtgcaccatgccggccacatccagagagctgtcatgggtggatgctgggcgcggggttctcttcagcagccggtgcaccatgccggccaggacttggcggaacggccggcggagcagagagtacgtgaacgggtcggccaccgccttgctgtaggtcaggcacttgctgaggatgccccactgggcgttcacggtgacgaagggcacgagctccgccagcctgttggggaagcggagcagctaactcagccacagtgacagcacctgccacgcactcacgctccgggggaaggtgctagaaccatactcaagtacataccgcagccctgatgctcactagccagggggctctgcaccagcaccctgagcctcgggctcctcttcctaaaatgggattacaaggcccagtcctgggactgcggtgcaagccgacatcagcccgcataccacacagcaggtgctcagtaggtggcagcactgccaccgtcaccaaggccttctcccaccatcctcacactcagctcagtccacccacctgcacgtacagctgccgcggacacctgcacacacaaccagctaccgagggagccattaccactaccaggctgcagaccaacacaccccagatgtgagctctgggagcccccaaccagactctcctctggcatttatctcccaaacctggacaatgttgagaacagatcagaacaatgctcataaccaggctggagtaacaggtgtcaacctgggctgcagaacagaccctgagcaagtgtgaccacctcacccacatgaacagcagtcactcacaggatgtccacgggcaccacacaccacgcagggtgcttccctacccactgtcccgaagcatcactcagagaagctcgagggtgtctctaggaccaccaggcacacaactcccataagaaaggctcagtctcacactctgtgtctgagcccagtgcttttctgaaatcagattagaactcagagtagaagggactgggctctgcgtgggggaaagcagcagggggatgggcctctggggactctgacggacccctccgctctcagcacagcctagggtaggggccatggagtcaggacaaggcttctgaccagcggcagcacagccttaggtgggggccacagagtgaggacgaggcttctgacagctgtcacaagcacacttgctgaccagtggcagctgttccacctcccagttcctggctttgaaccagggcccccaaaggccagggctttcccccacagcctgatgatctcccaggcagtgaatttctgtcccaggatgtcccacccccactcagaaggactgcagccctgtgatttacaagtttggctgggtcactcattagtttggaccacccacaaacacagcagctgctggggtgcagcaatggatcagactgactccctccctccgaggcacccagtccggcgggatggggatgaggaggctggcaggcaggcaggcaggcaaccacctcaagctgaggggcagggccagaatgtgacgacagagagaaggtgaggggcacagccagagcagaggcatccagaatccaaaaatcagggcagggacacagatccaaggcagtcatggctctaaccacctaccccaggctgtggtgaggatccagttacagtaagtgatagtccggaaagggctttgccaccggccagcacatgcacatcaggtacactgtcacccccacatacagggagcccaccatcaggcatagactggagactggtcacagccaaccatcaggcaagtgggaaggaaggtcctatcgaaggatctgaggaggctggcatttctttcagccactaggggactgggctgcaggtgagtaagaaagccacacacatgccagagtgtctatcccctagttaagccacggtgcaggtggaagtcatcagaaacacccagctgaggctgtgtctggagccacactgggaccagaactcatgtatcaactaccagccatgaccctgcagagaagagggctcgctcacgtgtccactgctgctcaacaccttgtgaccgtagccatctgccctcccccgtctccagtagaccacagactccacaagggagggctctgtatccctgccctcctggtctcactgccgcacaccccacgggcttcatctgcacgactgattgagtcagtaaaactcaagagtgacaaccaagaaacaaaataaaaatcccatgtcccccatctcctttacctactttatccgcatgaactggctcaaggtgacttggagttatttctaaccagattaaattcaccttctagatgtccacagaagagtctaacaatgcagcccagttctgaaaacaattcccaggagaagagctctgaaatatttttagcaattgcaggacaactggaatgagtgcactaccttttaaggggcagcagggacaacacactcctggatctctgacttcctgtgggttaagccaccaactgcattagaacctcatccacacacactcaggagtgaggttctgattcggttcactcctatgatgtgagaggcgcctgtgaggcaggtccgtccaaactggtccagctgtgctgctcccaggcgccagccctcaagacccctcccccactgaaacacctgcctggtcttgctgacctgcatctcccaccatcagaaccgagaggagcctgtgttccatttgcttttgtaactgcatgaccaggcacaaagcccagctcagagtaggtgctcattaaatgcttagtgagtgcatgaagaaatatataacctcgttagtgctatattgtacccacatttttcacagagaagagagcctgagctgcccagatcccagagttggtaacgacattgtggaaacaacactgcaaaggcaggcttcatgactttgtttgggattcttcctcttacactcgtgtgcacactcatagctatgcatgctcacatacatgttcagggcattgtaaacacacaggaaaataacagttacacccatgaatcaacataatatggctgagtaagccagtctcactcatcattcatttaaaatttcaaaactaaatgctggagctcttgaaattttagcactcaccttctacacatatgtacatgcatgtgcatgtatgaatacacacatgcatgtgtgtatacacatgaaccctcacaacatacacatgcatgcatgcgcaggcacatgggcacacatacacacaaacatgataatcagataaatagctggttcccgggagaaggatgctacagtttccccatccacacttgtgcccatgaagtttcctaagaaccagaggcctttgccaggttttatgtgaggaaggaggatgcctgtgatccacaagaaacctggctccctgtccctaagccagatgggatcagtggtgccaagagctcctgtcccacgtgctgattctgaagagctcagagggaacctggcaggcaggagagagctggtgccactcagcgtgggcactctggggcctcccgggttcctgggacacaactgtttcccccccacccacccaacaacatatctaagccacccagtaacctgggaagacaagcagtcatcctggacaggctggcagaccttgtagggcattagatagcaacctcaggaggaccctacacaaacagagccaggatcctggtactatactccctgctggctccctcccctggggtggggacctgggccccttgaggatgagggccatggacccctcccaggagcgtcttccttcagggtatcctcctgctatggctcaacacatagggtggagacctcccagacagcctgagcgcttcaggggcctggccagaaattccatggagattccaagaaaggcaaaacgtaatagcaagcagtcaccatctctaataaacaacctgcctccctctaaccacggtgccttccagcaaggcagggtgcacacatttccttgagtggagctaaggtagtaaccaagtggccgagtgggtgacccaggaaccccggaggcaaaaactacagactcccctgcccctcagcacagcactgtgatgatggtaccagcaggccctttacacacactctttcccaaattacctgtgactccaggtaattctgacctgacaagcactcaggccaaggtctccaggaaagagagaagcaaagagaacactcacctggcagtgttactgccccgaggctccagctggagcttccacccagattcccaactccacgcccaggagatgcggagtctgaggcctgagttagtcccaggttgcctgattttgaaagctttctaggtttaaagagccaggtgctgattcacagaccccatatctgcctgtgcctggacaaggagagtctcctcatttataggacaggatgggagccagtgccacctccctcccaggatctttcccaggagctgaagcacagccctgagcccgtcaggtgtggaggagggtcctctggtggtggccgccctccagatatcttgctgggaaacctcagaggcctactcaactgaggccaagcccacccagctgttcccacaacaggagccggactgccaggacccacctggtcatgacatacggggcaaagcagatgaggaaggtcgcaatagcaatgccaatcttcctggtggcgcggtggcggcgccgcttctgctggatgaggcagcgctgccgcacactgtggggacagaggacagtggtcaggatgatggcagggtggggacagaacccaggcccagagcccaggggctgaagtgagggcagacaggagggtttcaggaggatgtttagcctggaacctgaggaggacagagggatgggggagagagaaagaacaaggggtaggcttgcttttttgtaggtgaaataaaggaagtctggaaagaagggccagctcctcctcccttccctctggctcatccttctccacacagaggcaactgaggcaggctgctgatggaggggcaaggctggggccctcaaggagggagaagtcacgtggcctgcccaccccaggagcaggagagcctcttgggtgttctgtggggggccaagggggaaaggggaacacctacaggaggtaatttggaacaatggcgacaccagctagagattcaggagacaaatcctgccaagtagtgagcagaaagcatgggggatggaccctgctcaagacttgaagcccaagtccatgcaagggcactgggtcagcagtgggcagaaacaagcagatgcctcagacccacagccatgcacaggaggggtgtgtctggccgcccacagcagagcccgcggaacccctcccggagaagcacaccccacagctctgcgcaaagctccctgtgcactgtgagcacatatcccaggaggcagaggcccccgcagtgtctgcaccaatgaaccaacaggaaacccctgtgagcagcgtgcaggggcagtgctgtggctggtgaactctgggcagagtaacggtggtgatgcccacaggctcctcaagccaactgcccagggagcgcccaagtccctggcctgggcctgtcggcatgcactgggccaatacctggggtgcaggtcggcgagcagcgcgagcgccttcatggtgacggtgtccatgcgctggcagtgtctgcgtgccacccggtgcacctggagcgaggtgaggcagagcaccgccagcggcagcacgaagcccacggcatggagcgtggcggtgaaggctgcgaagcgcggacgctcaggctcgggcggcaggcgcagcgaacaggacgcgaaggcgctgctgtagccaagccacgagcagccaagtgcagcgcctgagaaggccagcgactgtccccaggcacagcccagcagcaggccggcatagcgcggtcgcaggcgtccggcgtagcgcagtgggaagcccactgccagccactggtctgcgctcagcgccgccacgctcagcgccgcgttggacgccaggaaggtgtccaggaagccaatgacttggcatgcgccgggcgccgacggtgtccgcccgcgcatcacaccgagcagcgtgaagggcatgtccagcgccgccagcagcaggtggcccagagacagattcaccaggaggacgcctgaggctcgagtgcggagctcagcgctgtaggcgcaacaaagcagcaccagtgcgttggatagcagcgccacggccagtaccatcaccaggagacccgccagcagcgcctcgccggggcccatggcgctagcggctcgccaggcaccctggggttctcatggctctgcttcgggcgcgagcctgggaaagtgaggcgatggagcagctgagcggcgcgcccacggcttctgggaggtgcagagccggctagcaaggtccagagcagcgcgaggaaagcggcggcaagtgcagggtccggggcacccggggggagggcgtagctctgcggagcaggagcagcccggggccgccctggaggcagcgcaaccgcggtgctgtccagggtgctgaatgcgctcaccgtcccggc

SEQ ID NO: 2 - DNA, 1092 bp, Homo sapiens
atgggccccggcgaggcgctgctggcgggtctcctggtgatggtactggccgtggcgctgctatccaacgcactggtgctgctttgttgcgcctacagcgctgagctccgcactcgagcctcaggcgtcctcctggtgaatctgtctctgggccacctgctgctggcggcgctggacatgcccttcacgctgctcggtgtgatgcgcgggcggacaccgtcggcgcccggcgcatgccaagtcattggcttcctggacaccttcctggcgtccaacgcggcgctgagcgtggcggcgctgagcgcagaccagtggctggcagtgggcttcccactgcgctacgccggacgcctgcgaccgcgctatgccggcctgctgctgggctgtgcctggggacagtcgctggccttctcaggcgctgcacttggctgctcgtggcttggctacagcagcgccttcgcgtcctgttcgctgcgcctgccgcccgagcctgagcgtccgcgcttcgcagccttcaccgccacgctccatgccgtgggcttcgtgctgccgctggcggtgctctgcctcacctcgctccaggtgcaccgggtggcacgcagacactgccagcgcatggacaccgtcaccatgaaggcgctcgcgctgctcgccgacctgcaccccagtgtgcggcagcgctgcctcatccagcagaagcggcgccgccaccgcgccaccaggaagattggcattgctattgcgaccttcctcatctgctttgccccgtatgtcatgaccaggctggcggagctcgtgcccttcgtcaccgtgaacgcccagtggggcatcctcagcaagtgcctgacctacagcaaggcggtggccgacccgttcacgtactctctgctccgccggccgttccgccaagtcctggccggcatggtgcaccggctgctgaagagaaccccgcgcccagcatccacccatgacagctctctggatgtggccggcatggtgcaccagctgctgaagagaaccccgcgcccagcgtccacccacaacggctctgtggacacagagaatgattcctgcctgcagcagacacactga

SEQ ID NO: 3 - protein, 363 aa, Homo sapiens
MGPGEALLAGLLVMVLAVALLSNALVLLCCAYSAELRTRASGVLLVNLSLGHLLLAALDMPFTLLGVMRGRTPSAPGACQVIGFLDTFLASNAALSVAALSADQWLAVGFPLRYAGRLRPRYAGLLLGCAWGQSLAFSGAALGCSWLGYSSAFASCSLRLPPEPERPRFAAFTATLHAVGFVLPLAVLCLTSLQVHRVARRHCQRMDTVTMKALALLADLHPSVRQRCLIQQKRRRHRATRKIGIAIATFLICFAPYVMTRLAELVPFVTVNAQWGILSKCLTYSKAVADPFTYSLLRRPFRQVLAGMVHRLLKRTPRPASTHDSSLDVAGMVHQLLKRTPRPASTHNGSVDTENDSCLQQTH